(19) United States
(12) Patent Application Publication
(10) Pub. No.: US 2014/0174948 A1
(43) Pub. Date: Jun. 26, 2014

(54) METHOD OF A TEST STRIP DETECTING CONCENTRATION OF AN ANALYTE OF A SAMPLE, THREE-ELECTRODE TEST STRIP, AND METHOD OF UTILIZING A TEST STRIP DETECTING DIFFUSION FACTOR

(71) Applicant: TYSON BIORESEARCH INC., Miaoli Hsien (TW)

(72) Inventors: Cheng-Che Lee, Hsinchu County (TW); Wen-Huang Chen, New Taipei City (TW); Han-Ching Tsai, New Taipei City (TW); Chen-Yu Yang, Miaoli County (TW)

(73) Assignee: TYSON BIORESEARCH INC., Miaoli Hsien (TW)

(21) Appl. No.: 14/138,110

(22) Filed: Dec. 22, 2013

Related U.S. Application Data
(60) Provisional application No. 61/745,644, filed on Dec. 23, 2012.

(30) Foreign Application Priority Data
Dec. 9, 2013 (TW) 102145169

(51) Int. Cl. C12Q 1/00

(52) U.S. Cl. CPC C12Q 1/001
USPC 205/777.5; 204/403.04

(57) ABSTRACT

A method of a test strip detecting concentration of an analyte of a sample includes placing the sample in a reaction region of the test strip, wherein the analyte reacts with an enzyme to generate a plurality of electrons, and the plurality of electrons are transferred to a working electrode of the reaction region through a mediator; applying an electrical signal to the working electrode; measuring a first current through the working electrode during a first period; the mediator generating an intermediate according to the electrical signal during a second period; measuring a second current through the working electrode during a third period; calculating initial concentration of the analyte according to the first current; calculating a diffusion factor of the intermediate in the sample according to the second current; and correcting the initial concentration to generate new concentration of the analyte according to the diffusion factor.

### CROSS REFERENCE TO RELATED APPLICATIONS

This application claims the benefit of U.S. Provisional Application No. 61/745,644, filed on Dec. 23, 2012 and entitled “Biosensors and Test Strips for Improving Measurement Accuracy and Methods for Same,” the contents of which are incorporated herein by reference.

### BACKGROUND OF THE INVENTION

1. Field of the Invention

The present invention relates to method of a test strip detecting concentration of an analyte of a sample, a test strip with three electrodes, and a method of utilizing a test strip to detect a diffusion factor of a mediator in a sample, and particularly to a method that can utilize an electrical signal having different polarities during different periods to detect concentration of an analyte of a sample or to detect a diffusion factor of a mediator in a sample, and a test strip that can utilize an electrical signal having different polarities during different periods to detect concentration of an analyte of a sample or to detect a diffusion factor of a mediator in a sample.

2. Description of the Prior Art

Electrochemical biosensors have been commonly used to determine the concentration of various analytes from test samples, such as glucose, uric acid and cholesterol in biological fluids. For example, in biological sample testing, the test strip may be inserted into a glucose meter, and a fluid sample is dropped on a test strip and introduced into a sample chamber to determine the concentration of the analyte in the biological sample.

In the recent years, people with diabetes are growing. Glucose concentration monitoring is important of everyday life for diabetic patents. Routine tests for 3-4 times every day and controlling stabile blood glucose concentration can reduce the risk of serious damages, such as vision loss and kidney failure. The accurate measurement of blood glucose is expected.

However, biosensors may provide the testing results including the multiple analytic errors. When the testing sample is a whole blood, these error sources may come from physical characteristics of the whole blood (e.g. interferences), environmental factors (e.g. temperature), and operating conditions (e.g. under-fill). The physical characteristics of the blood include interferences, such as hematocrit (ratio of red blood cell volume to the total blood volume), ascorbic acid, uric acid, cholesterol, and the like.

For example, the normal hematocrit range for a typical human is about 35% to 55%. However, in some special cases, the hematocrit may range from 10% to 70% and induce the large error in the blood glucose measurement. At high hematocrit, the red blood cell may hinder the reaction of enzyme and mediators, and even reduce the diffusion rate of the mediators to the working electrode, resulting in the low blood glucose reading. Conversely, the low hematocrit may result in the high blood glucose reading.

There are many methods to minimize the analytic error of the hematocrit effect. For example, U.S. Pat. No. 5,951,836 disclosed a reagent formulation using silica particle to filter red blood cell. U.S. Pat. No. 5,628,890 disclosed reducing the hematocrit effect by using wide spacing in combination with mesh layers to distribute the blood sample. U.S. Pat. No. 8,388,821 disclosed a method for hematocrit correlated measurement by providing a plurality of microelectrodes on a working electrode. U.S. Pub. No. 2011/0139634 disclosed hematocrit-corrected analyte concentration by using two electrode sets, which applying DC signal and AC signal, separately. The prior methods had some disadvantage, such as high manufacturing cost, a complex process and a large sample amount.

Besides, temperature during the measurement is another analytic error source. Since the enzyme reaction is a temperature-dependent reaction, the temperature change during the measurement has an influence on the measurement accuracy.

To sum up, the above mentioned methods provided by the prior art are not good choices for a user.

### SUMMARY OF THE INVENTION

An embodiment provides a method of a test strip detecting concentration of an analyte of a sample, wherein the test strip includes a substrate and a reaction region, the reaction region comprises a working electrode, a reference electrode, and a counter electrode, and an enzyme is coated in the reaction region. The method includes placing the sample in the reaction region, wherein the analyte reacts with the enzyme to generate a plurality of electrons, and the plurality of electrons are transferred to the working electrode through a mediator; applying an electrical signal to the working electrode; measuring a first current through the working electrode during a first period; the mediator generating an intermediate according to the electrical signal during a second period; measuring a second current through the working electrode during a third period, wherein a second polarity of the electrical signal during the second period is inverse to a first polarity of the electrical signal during the first period and a third polarity of the electrical signal during the third period; calculating initial concentration of the analyte according to the first current; calculating a diffusion factor of the intermediate in the sample according to the second current; and correcting the initial concentration to generate new concentration of the analyte according to the diffusion factor.

Another embodiment provides a test strip with three electrodes. The test strip includes a substrate and a reaction region. The reaction region is formed on a first end of the substrate, and an enzyme is coated in the reaction region, wherein when a sample is placed in the reaction region, an analyte reacts with the enzyme to generate a plurality of electrons, and the plurality of electrons are transferred through a mediator. The reaction region includes a working electrode, a reference electrode, and a counter electrode. The working electrode is used for receiving an electrical signal when the sample is placed in the reaction region, generating a first current according to the electrical signal during a first period, and generating a second current according to the electrical signal during a third period behind a second period, wherein a second polarity of the electrical signal during the second period is inverse to a first polarity of the electrical signal during the first period and a third polarity of the electrical signal during the third period, wherein the mediator generates an intermediate according to the electrical signal during the second period. The reference electrode is used for receiving a reference voltage when the sample is placed in the reaction region. The counter electrode is used for receiving a floating voltage when the sample is placed in the reaction region to satisfy a current generated by the working electrode during the first period, the second period, and the third period, wherein the current comprises the first current and the second current. The first current is used for calculating initial concentration of the analyte, the second current is used for calculating a diffusion factor of the intermediate, and the diffusion factor is used for correcting the initial concentration to generate new concentration of the analyte.

Another embodiment provides a method of a test strip detecting concentration of an analyte of a sample, wherein the test strip includes a substrate and a reaction region, the reaction region comprises a working electrode, a reference electrode, and a counter electrode, and an enzyme is coated in the reaction region. The method includes placing the sample in the reaction region, wherein the analyte reacts with the enzyme to generate a plurality of electrons, and the plurality of electrons are transferred to the working electrode through a mediator; applying an electrical signal to the working electrode; measuring a first current through the working electrode during a first period; the mediator generating an intermediate according to the electrical signal during a second period; measuring a second current through the working electrode during a third period, wherein the electrical signal has a second polarity and a non-polarity during the second period, and the second polarity is inverse to a first polarity of the electrical signal during the first period and a third polarity of the electrical signal during the third period; calculating initial concentration of the analyte according to the first current; calculating a diffusion factor of the intermediate in the sample according to the second current; and correcting the initial concentration to generate new concentration of the analyte according to the diffusion factor.

Another embodiment provides a method of utilizing a test strip to detect a diffusion factor of a mediator in a sample, wherein the test strip includes a reaction region, and the reaction region includes a working electrode, a reference electrode, and a counter electrode. The method includes placing the sample in the reaction region; applying an electrical signal to the working electrode; the mediator generating an intermediate according to the electrical signal during a first period; measuring a first current through the working electrode during a second period behind the first period, wherein a second polarity of the electrical signal during the second period is inverse to a first polarity of the electrical signal during the first period; and calculating the diffusion factor of the intermediate in the sample according to the first current.

Another embodiment provides a method of utilizing a test strip to detect a diffusion factor of a mediator in a sample, wherein the test strip includes a reaction region, and the reaction region includes a working electrode, a reference electrode, and a counter electrode. The method includes placing the sample in the reaction region; applying an electrical signal to the working electrode; the mediator generating an intermediate according to the electrical signal during a first period; measuring a first current through the working electrode during a second period behind the first period, wherein the electrical signal has a first polarity and a non-polarity during the first period, and the first polarity is inverse to a second polarity of the electrical signal during the second period; and calculating the diffusion factor of the intermediate in the sample according to the first current.

The present invention provides a method of a test strip detecting concentration of an analyte of a sample and a test strip with three electrodes. The method and the test strip utilize a working electrode to generate a first current for calculating initial concentration of the analyte of the sample according to an electrical signal provided by a meter during a first period, utilize the working electrode to make the mediator in the sample generate reaction according to the electrical signal during a second period, and utilize the working electrode to generate a second current for calculating a diffusion factor of the mediator in the sample according to the electrical signal provided by the meter during a third period. After the diffusion factor of the mediator in the sample is generated, the meter can correct the initial concentration of the analyte in the sample to generate new concentration of the analyte according to the diffusion factor. Therefore, compared to the prior art, the present invention can accurately correct the initial concentration of the analyte in the sample. In addition, a method of utilizing a test strip to detect a diffusion factor of a mediator in a sample further provided by the present invention utilizes a second polarity of an electrical signal during a second period inverse to a first polarity of the electrical signal during a first period to detect the diffusion factor of the mediator. Therefore, compared to the prior art, the present invention can rapidly, simply, and accurately to detect the diffusion factor of the mediator.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is an explosion diagram illustrating a test strip according to an embodiment.

FIG. 2 is a diagram illustrating a cross-section of the test strip.

FIG. 3 is a diagram illustrating a test strip with three electrodes.

FIG. 4 is a diagram illustrating a test strip with three electrodes.

FIG. 5 is a diagram illustrating a test strip with three electrodes, a reaction region, and a meter according to another embodiment.

FIG. 6 is a diagram illustrating a test strip with three electrodes, a reaction region, and a meter according to another embodiment.

FIG. 7 is a diagram illustrating a test strip with three electrodes, a reaction region, and a meter according to another embodiment.

FIG. 8 is a diagram illustrating a structure of the test strip.

FIG. 9 is a diagram illustrating a test strip with three electrodes according to another embodiment.

FIG. 10 is a diagram illustrating the voltage drop between the working electrode and the reference electrode during a first period, a second period, and a third period.

FIG. 11 to FIG. 14 are diagrams illustrating distribution of a mediator and an intermediate in the reaction region being changed with the voltage drop between the working electrode and the reference electrode when the sample is placed in the reaction region.

FIG. 15 to FIG. 17 are diagrams illustrating relationships between the diffusion factor of the mediator and the current generated by the working electrode under different interfering substances.

FIG. 18 to FIG. 24 are diagrams illustrating a voltage drop between the working electrode and the reference electrode during the first period, the second period, and the third period according to different embodiments.

FIG. 25 is a diagram illustrating currents generated by the working electrode corresponding to different samples under the voltage drop between the working electrode and the reference electrode in FIG. 10.

FIG. 26 is a diagram illustrating relationships between biases and concentration of analytes with different hematocrits not corrected by the present invention.

FIG. 27 is a diagram illustrating relationships between biases and concentration of analytes with different hematocrits corrected by the present invention.

FIG. 28 is a flowchart illustrating a method of a test strip detecting concentration of an analyte of a sample according to another embodiment.

FIG. 29 is a flowchart illustrating a method of utilizing a test strip to detect diffusion factor of a mediator in a sample according to another embodiment.

### DETAILED DESCRIPTION

The present invention will now be described through the following embodiments. It is understood by those skilled in the art that the embodiments described below are only for illustration purpose and no limitation of scope of the invention is intended.

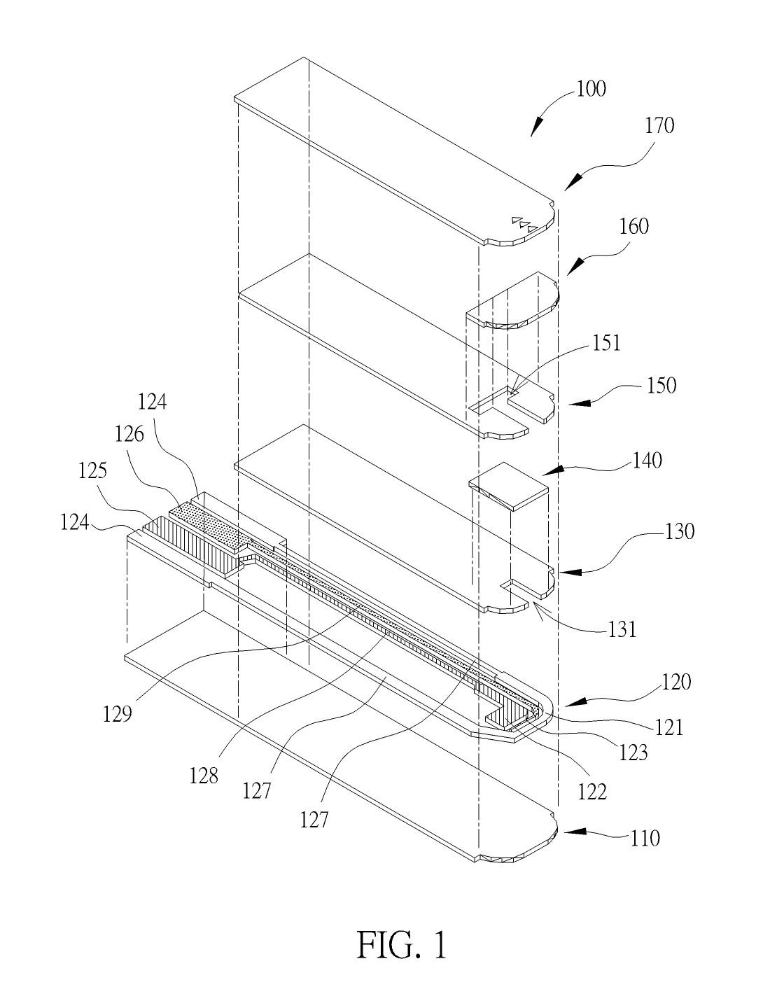

Please refer to FIG. 1. FIG. 1 is an explosion diagram illustrating a test strip 100 according to an embodiment. As shown in FIG. 1, the test strip 100 may include a substrate 110, an electrode layer 120, an insulating layer 130, a reagent 140, a spacer 150, a first cover layer 160, and a second cover layer 170, wherein the substrate 110 can be made of a plastic material (e.g. polyethylene terephthalates (PET), vinyl polymers, polyimides, or polyesters).

As shown in FIG. 1, the substrate 110 carries the electrode layer 120, wherein electrode layer 120 has a working electrode 121, a counter electrode 122, and a reference electrode 123, and the working electrode 121, the counter electrode 122, and the reference electrode 123 are formed on a first end of the electrode layer 120. The working electrode 121, the counter electrode 122, and the reference electrode 123 can be formed by laser scribing on the conductive electrode layer 120 or by screen printing onto the substrate 110. A second end of the electrode layer 120 can provide a plurality of pads 124, 125, 126, wherein the plurality of pads 124, 125, 126 are used for electrical coupling to a meter. An electrode track 127 can provide an electrically continuous pathway from the working electrode 121 to the pad 124. Similarly, an electrode track 128 provides an electrically continuous pathway from the counter electrode 122 to the pad 125, and an electrode track 129 provides an electrically continuous pathway from the reference electrode 123 to the pad 126. The electrode layer 120 can be made of conductive materials provided by the prior art (e.g. gold, platinum, sliver, carbon and carbon/silver).

An insulating layer 130 can be used for protecting the electrode tracks 127, 128, 129 and defining an effective area of a reaction region. A notch 131 maybe located on a front section of the insulating layer 130 for exposing portions of the working electrode 121, the counter electrode 122, and the reference electrode 123, wherein the exposed portions of the working electrode 121, the counter electrode 122, and the reference electrode 123 and the reagent 140 can be combined to form the reaction region. The insulating layer 130 can be made of ink, ultraviolet radiation (UV) polymer or the like, and can be formed on the electrode layer 120 through the screen printing.

The reagent 140 may be located on the exposed portions of the working electrode 121, the counter electrode 122, and the reference electrode 123 exposed by the notch 131. The choice of the reagent 140 depends on the specific analyte and is well known by those skilled in the art. In one embodiment of the present invention, the reagent 140 is used for measuring glucose from a human blood sample. A non-limiting reagent can include an enzyme, electron mediators, stabilizers and binders, wherein the enzyme can be glucose oxidase or glucose dehydrogenase. The electron mediator is an electron acceptor and capable of transferring electrons between the enzyme and the working electrode 121. Typically, the mediator can be ferrocene, potassium ferricyanide or other ferrocene derivatives. In one embodiment of the present invention, the reaction region at the reagent 140 is where the glucose in the human blood sample reacts with the enzyme, electrons are transferred through the mediators to the working electrode 121, and an electrical response is generated.

The spacer 150 overlies the substrate 110 and can define height of the sample-receiving chamber. In one embodiment of the present invention, the spacer 150 has a T-shaped channel 151 located at a front section of the spacer 150.

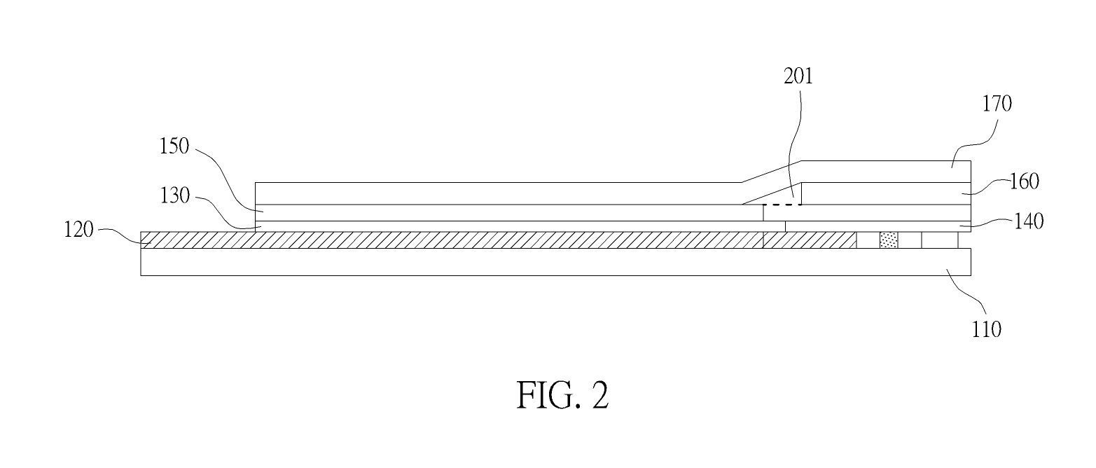

The first cover layer 160 is attached on a part of the spacer 150 for forming a top surface of the sample-receiving chamber. A bottom of the first cover layer 160 includes a hydrophilic coating (not shown in FIG. 1). When the human blood sample enters the sample-receiving chamber, the hydrophilic coating can help a capillary action and increase a speed of movement of the human blood sample. As shown in FIG. 1, a final layer of the test strip 100 is the second cover layer 170. The second cover layer 170 includes a transparent window that allows a user to visually confirm if the human blood sample enters the sample-receiving chamber. As shown in FIG. 2, the first cover layer 160, the second cover layer 170, and the spacer 150 form a opening 201, wherein when the human blood sample enters the sample-receiving chamber, the opening 201 allows air to escape from the interior of the sample-receiving chamber.

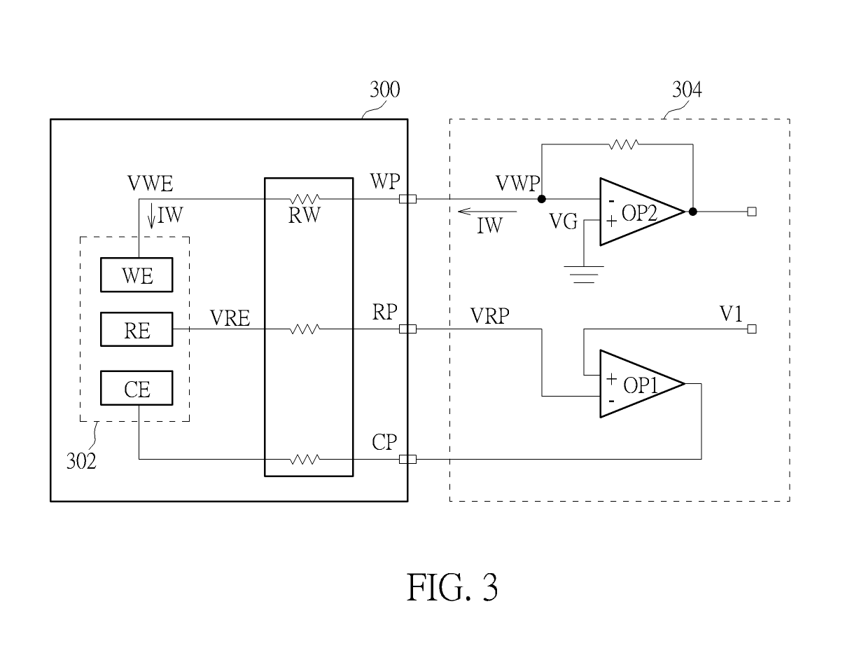

In general biochemical measurements, a meter applies an electrical signal to a working electrode of a test strip, and then the meter measures a current generated by the working electrode for the following measurements. Please refer to FIG. 3. FIG. 3 is a diagram illustrating a test strip 300 with three electrodes, a reaction region 302, and a meter 304, wherein the meter 304 is electrically connected to a working electrode WE, a reference electrode RE, and a counter electrode CE of the reaction region 302 through pads WP, RP, CP, respectively, and OP1, OP2 are operational amplifiers. As shown in FIG. 3, when a sample is placed in the reaction region 302 of the test strip 300 coupled to the meter 304, the meter 304 can apply a fixed voltage (e.g. ground potential VG) to the operational amplifier OP2 to measure a current IW generated by the working electrode WE. Because an electrode track between the working electrode WE and the pad WP has an equivalent resistor RW, a real voltage VWE of the working electrode WE can be determined by equation (1):

$$VWP = VG \\ \begin{matrix}VWE = VWP - IW \times RW \\ = VG - IW \times RW\end{matrix} \qquad ( 1 )$$

In addition, a voltage drop VWR (equal to the electrical signal applied to the working electrode WE) between the working electrode WE and the reference electrode RE can be determined by equation (2):

$$\begin{matrix}VWR = VWE - VRE \\ = VG - IW \times RW - VRE \\ = VG - VRP - IW \times RW \\ = VG - V 1 - IW \times RW\end{matrix} \qquad ( 2 )$$

As shown in equation (2), VRE is a voltage of the reference electrode RE, VRP is a voltage of the pad RP, and V1 is a reference voltage inputted to the operational amplifier OP1. As shown in equation (2), the voltage drop VWR between the working electrode WE and the reference electrode RE is influenced by the current IW. Because the voltage drop VWR between the working electrode WE and the reference electrode RE is influenced by the current IW, the meter 304 can provide a stable voltage to the reference electrode RE, but can not provide a stable voltage to the working electrode WE for the accurate subsequent measurements. Therefore, the test strip with three electrodes (e.g. the test strip 300 as shown in FIG. 3) provided by the prior art can not meet requirements of a user.

Please refer to FIG. 4. FIG. 4 is a diagram illustrating a test strip 600 with three electrodes, a reaction region 602, and a meter 604 according to an embodiment, wherein the meter 604 is electrically connected to a working electrode WE of the reaction region 602 through pads WP1, WP2, a reference electrode RE of the reaction region 602 through a pad RP, and a counter electrode CE of the reaction region 602 through a pad CP. In addition, OP1, OP2 in the meter 604 are operational amplifiers, and an enzyme is coated in the reaction region 602. As shown in FIG. 4, when a sample is placed in the reaction region 602 of the test strip 600 coupled to the meter 604, the meter 604 can apply a voltage V2 to the operational amplifier OP2 to measure a current IW generated by the working electrode WE, wherein the sample covers at least the working electrode WE. Because an electrode track between the working electrode WE and the pad WP2 has an equivalent resistor RW, a real voltage VWE of the working electrode WE can be determined by equation (3):

$$\begin{matrix}VWE = VWP - IWP \times RW \\ = VWP ( ∵ IWP = 0 ) \\ = V 2\end{matrix} \qquad ( 3 )$$

As shown in equation (3), VWP is a voltage of the pad WP2, and the IWP is a current following through the pad WP2. In addition, in FIG. 4, a voltage drop VWR between the working electrode WE and the reference electrode RE (equal to an electrical signal applied to the working electrode WE) can be determined by equation (4):

$$\begin{matrix}VWR = VWE - VRE \\ = VWP - VRP \\ = V 2 - V 1\end{matrix} \qquad ( 4 )$$

As shown in equation (4), VRE is a voltage of the reference electrode RE, VRP is a voltage of the pad RP, and V1 is a reference voltage inputted to the operational amplifier OP1. As shown in equation (4), the voltage drop VWR between the working electrode WE and the reference electrode RE (equal to the electrical signal applied to the working electrode WE) is not influenced by the current IW. Because the voltage drop VWR between the working electrode WE and the reference electrode RE is not influenced by the current IW, the meter 604 not only can provide a stable voltage to the reference electrode RE, but can also provide a stable voltage to the working electrode WE for the accurate subsequent measurements. Therefore, the test strip with 4 pads (e.g. the test strip 600 as shown in FIG. 4) provided by the present invention can meet the requirements of the user.

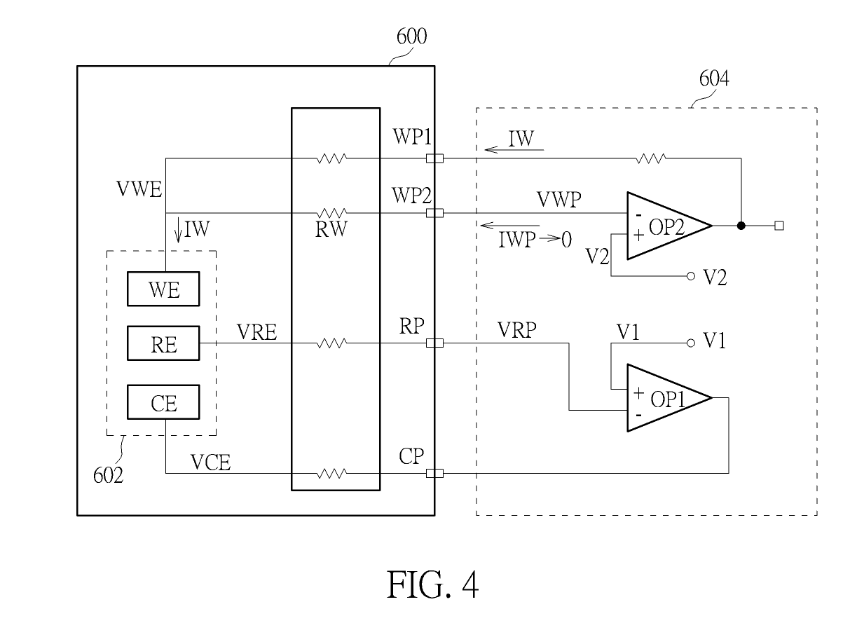

Please refer to FIG. 5. FIG. 5 is a diagram illustrating a test strip 600 with three electrodes, a reaction region 602, and a meter 704 according to another embodiment, wherein the meter 704 is electrically connected to a working electrode WE of the reaction region 602 through pads WP1, WP2, a reference electrode RE of the reaction region 602 through a pad RP, and a counter electrode CE of the reaction region 602 through a pad CP. In addition, OP1, OP2 in the meter 704 are operational amplifiers. As shown in FIG. 5, a difference between the meter 704 and the meter 604 in FIG. 4 is that a voltage V2 applied to the operational amplifier OP2 by the meter 704 is a variable voltage and a voltage V1 applied to the operational amplifier OP1 by the meter 704 is a fixed voltage. In addition, subsequent operational principles of the test strip 600, the reaction region 602, and the meter 704 in FIG. 5 are the same as those of the test strip 600, the reaction region 602, and the meter 604 in FIG. 4, so further description thereof is omitted for simplicity.

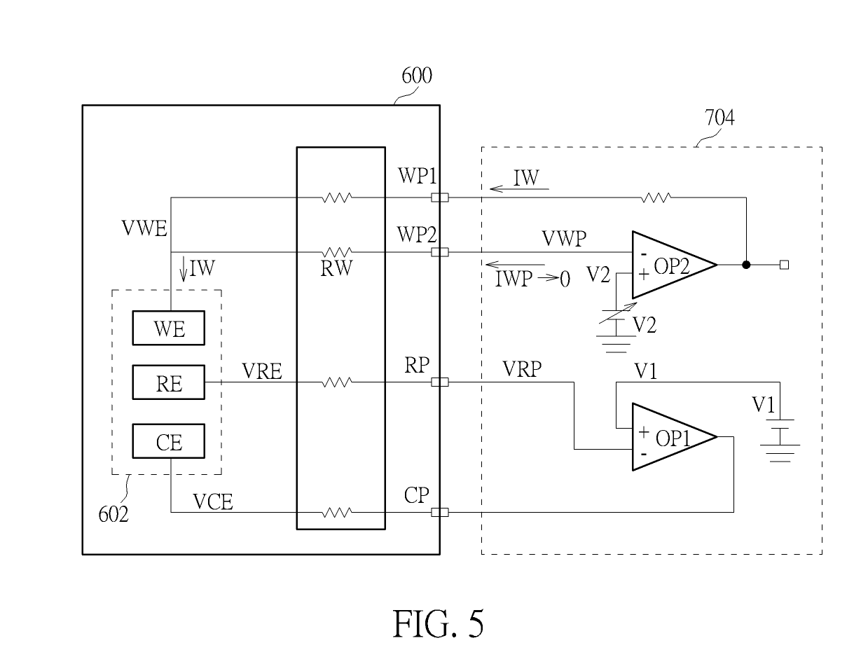

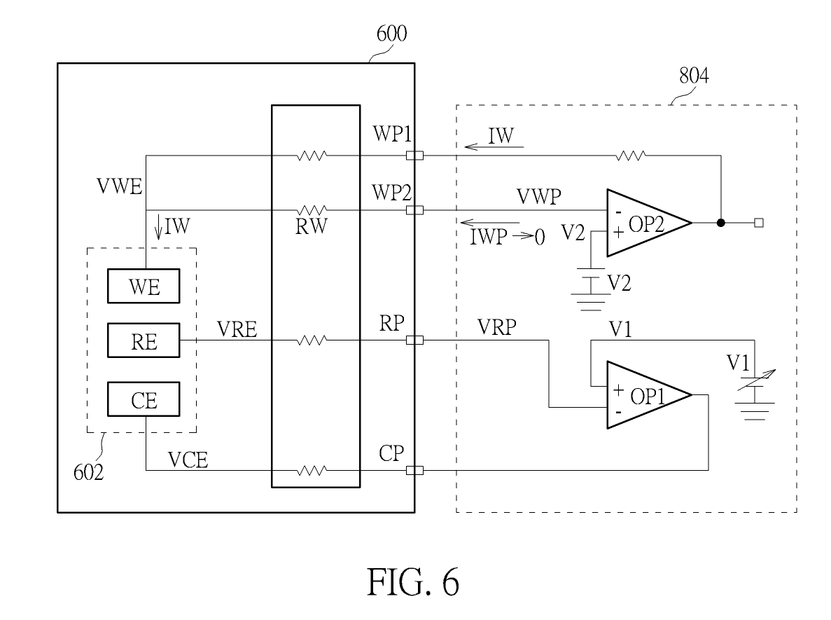

Please refer to FIG. 6. FIG. 6 is a diagram illustrating a test strip 600 with three electrodes, a reaction region 602, and a meter 804 according to another embodiment, wherein the meter 804 is electrically connected to a working electrode WE of the reaction region 602 through pads WP1, WP2, a reference electrode RE of the reaction region 602 through a pad RP, and a counter electrode CE of the reaction region 602 through a pad CP. In addition, OP1, OP2 in the meter 804 are operational amplifiers. As shown in FIG. 6, a difference between the meter 804 and the meter 604 in FIG. 4 is that a voltage V2 applied to the operational amplifier OP2 by the meter 804 is a fixed voltage and a voltage V1 applied to the operational amplifier OP1 by the meter 804 is a variable voltage. In addition, subsequent operational principles of the test strip 600, the reaction region 602, and the meter 804 in FIG. 6 are the same as those of the test strip 600, the reaction region 602, and the meter 604 in FIG. 4, so further description thereof is omitted for simplicity.

Please refer to FIG. 7. FIG. 7 is a diagram illustrating a test strip 600 with three electrodes, a reaction region 602, and a meter 904 according to another embodiment, wherein the meter 904 is electrically connected to a working electrode WE of the reaction region 602 through pads WP1, WP2, a reference electrode RE of the reaction region 602 through a pad RP, and a counter electrode CE of the reaction region 602 through a pad CP. In addition, OP1, OP2 in the meter 904 are operational amplifiers. As shown in FIG. 7, a difference between the meter 904 and the meter 604 in FIG. 4 is that a voltage V2 applied to the operational amplifier OP2 by the meter 904 is a variable voltage and a voltage V1 applied to the operational amplifier OP1 by the meter 904 is a variable voltage. In addition, subsequent operational principles of the test strip 600, the reaction region 602, and the meter 904 in FIG. 7 are the same as those of the test strip 600, the reaction region 602, and the meter 604 in FIG. 4, so further description thereof is omitted for simplicity.

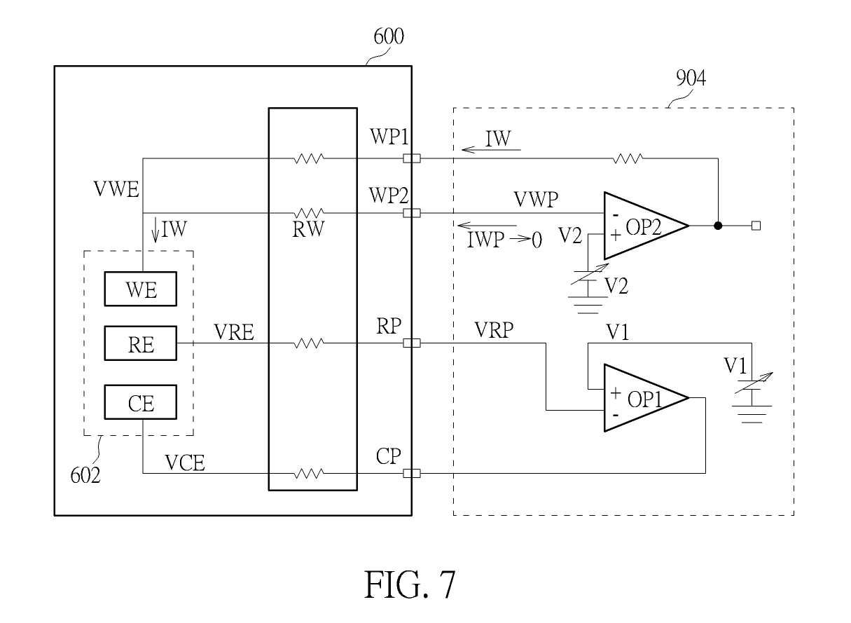

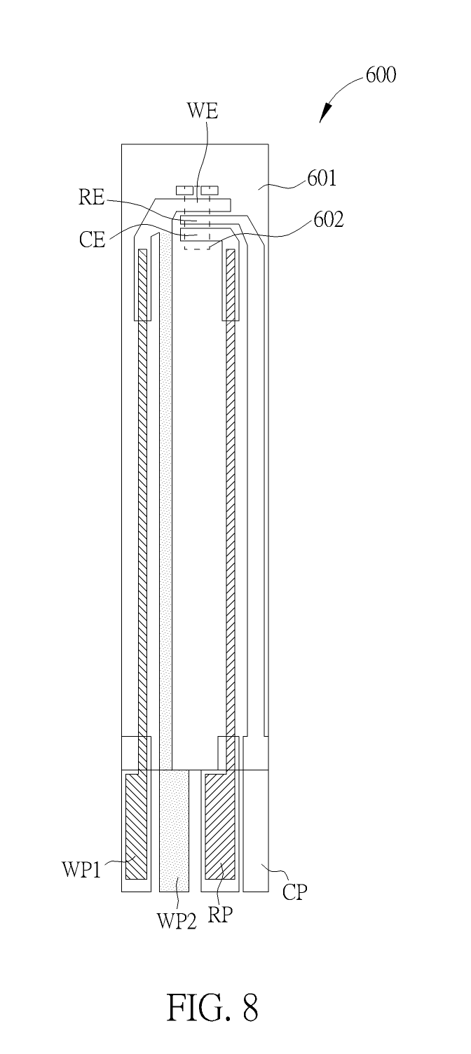

Please refer to FIG. 8. FIG. 8 is a diagram illustrating a structure of the test strip 600. As shown in FIG. 8, the test strip 600 includes a substrate 601, the reaction region 602, the working electrode WE, the reference electrode RE, and the counter electrode CE, wherein the working electrode WE connects to the pads WP1, WP2, the reference electrode RE connects to the pad RP, and the counter electrode CE connects to the pad CP. In addition, the substrate 601 is made of an insulting material (e.g. polyethylene terephthalate (PET) or insulting materials as like). As shown in FIG. 8, the working electrode WE, the reference electrode RE, and the counter electrode CE are formed on the substrate 601 and the reaction region 602 is formed on a first end of the substrate 601, wherein the working electrode WE, the reference electrode RE, and the counter electrode CE are made of conductive materials, wherein the conductive materials include gold, platinum, silver or graphite. But, the present invention is not limited to the conductive materials including gold, platinum, silver or graphite. In addition, the pads WP1, WP2, RP, CP are formed on a second end of the substrate 601, wherein the second end of the substrate 601 is opposite to the first end of the substrate 601. As shown in FIG. 8, the pads WP1, WP2 are formed on a left side of the substrate 601 (wherein positions of the pads WP1, WP2 can be changed each other), the pads RP, CP are formed on a right side of the substrate 601, and the pad RP is located between the pad WP2 and the pad CP. In addition, in another embodiment of the present invention, the pads WP1, WP2 are formed on the right side of the substrate 601, the pads RP, CP are formed on the left side of the substrate 601, and the pad CP is located between the pad WP2 and the pad RP. In addition, in the reaction region 602, the reference electrode RE is located between the counter electrode CE and the working electrode WE.

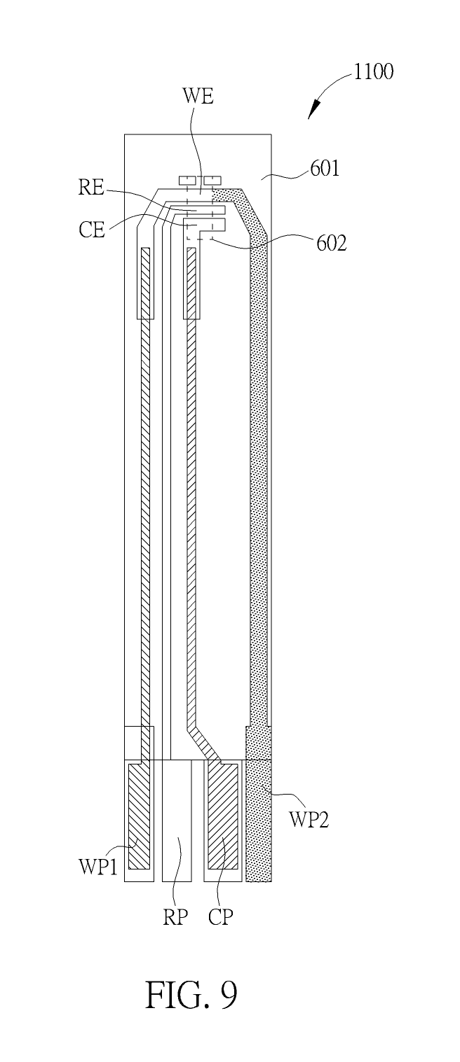

In addition, please refer to FIG. 9. FIG. 9 is a diagram illustrating a test strip 1100 with three electrodes according to another embodiment. As shown in FIG. 9, a difference between the test strip 1100 and the test strip 600 in FIG. 8 is that a pad WP1 and a pad WP2 coupled to a working electrode WE are formed on a left side and a right side of a substrate 601 respectively, a pad RP coupled to a reference electrode RE and a pad CP coupled to a counter electrode CE are formed on a middle of the substrate 601, and the pad RP is located between the pad WP1 and the pad CP. In addition, subsequent operational principles of the test strip 1100 are the same as those of the test strip 600, so further description thereof is omitted for simplicity.

In addition, as shown in FIG. 4, when a sample (e.g. blood) is placed in the reaction region 602 of the test strip 600 coupled to the meter 604 through the pads WP1, WP2, RP, CP, the meter 604 applies the voltage V2 to the operational amplifier OP2 and the reference voltage V1 to the operational amplifier OP1 to measure the current IW of the working electrode WE. As shown in equation (4), when the sample is placed in the reaction region 602 of the test strip 600, because the voltage drop VWR between the working electrode WE and the reference electrode RE (equal to the electrical signal applied to the working electrode WE) is not influenced by the current IW, the meter 604 can provide the stable voltage drop VWR and accurately measure the current IW generated by the working electrode WE through the pad WP1 for subsequent calculation. In addition, subsequent operational principles of the meter 704, the meter 804, and the meter 904 are the same as those of the meter 604, so further description thereof is omitted for simplicity.

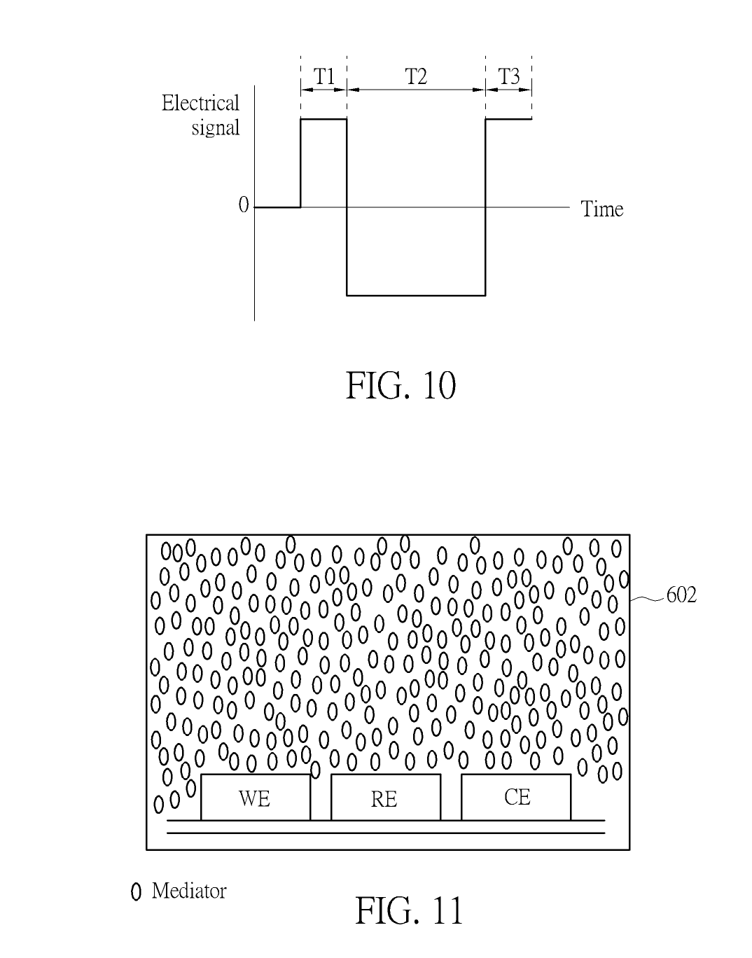

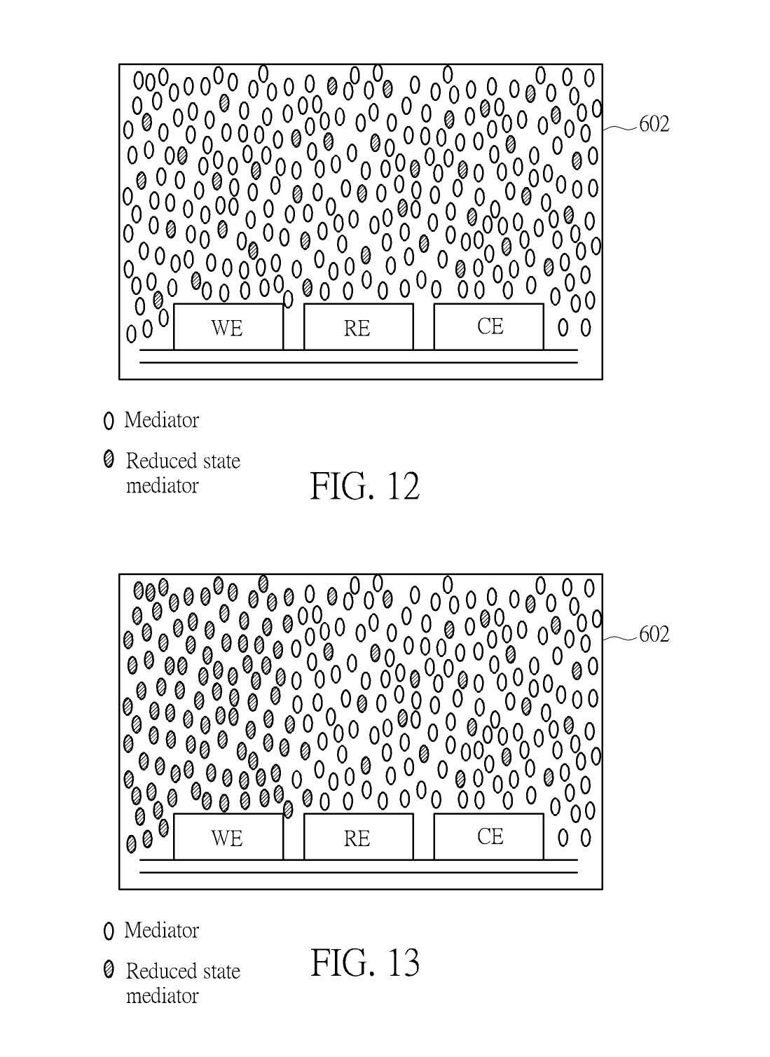

Please refer to FIG. 10 to FIG. 14. FIG. 10 is a diagram illustrating the voltage drop VWR between the working electrode WE and the reference electrode RE (equal to the electrical signal applied to the working electrode WE) during a first period T1, a second period T2, and a third period T3, and FIG. 11 to FIG. 14 are diagrams illustrating distribution of a mediator and an intermediate in the reaction region 602 being changed with the voltage drop VWR between the working electrode WE and the reference electrode RE when the sample (e.g. blood) is placed in the reaction region 602, wherein the mediator can be pre-coated in the reaction region 602 or be added to the reaction region 602 when the sample is placed in the reaction region 602. As shown in FIG. 10 and FIG. 11, when the sample (including an analyte (e.g. blood sugar)) is placed in the reaction region 602, the mediator in the sample can directly or indirectly seize electrons from the analyte to become a reduced state mediator, wherein concentration of the mediator is much greater than concentration of the analyte (e.g. the concentration of the mediator is equal to 2-4 times the concentration of the analyte). Therefore, as shown in FIG. 10 and FIG. 12, because the electrical signal applied to the working electrode WE is a positive polarity signal during the first period T1, the reduced state mediator can transfer electrons to the working electrode WE through a diffusion effect. That is to say, the working electrode WE can generate the current IW (first current) through the reduced state mediator during the first period T1, wherein the current IW (first current) during the first period T1 can be used for calculating initial concentration of the analyte. For example, if the sample is blood and the analyte in the sample is blood sugar, the mediator in the reaction region 602 can be potassium ferricyanide, wherein the potassium ferricyanide can directly or indirectly react with the blood sugar through the enzyme to generate reduced state potassium ferrocyanide. During the first period T1, the positive polarity signal applied to the working electrode WE can make the potassium ferrocyanide diffuse to the working electrode WE to generate the current IW (first current). But, the present invention is not limited to the mediator in the sample directly or indirectly seizing electrons from the analyte to be a reduced state mediator. That is to say, the mediator in the sample can also directly or indirectly transfer electrons to the analyte to become an oxidized state mediator.

As shown in FIG. 10 and FIG. 13, because the concentration of the mediator is much greater than the concentration of the analyte, the majority of mediator not reacted with the analyte can generate reduction reaction on a surface of the working electrode WE when the electrical signal applied to the working electrode WE is a negative polarity signal during the second period T2, resulting in the high concentration reduced state mediator (that is, the intermediate) being accumulated on the surface of the working electrode WE, wherein the concentration of the reduced state mediator accumulated on the surface of the working electrode WE is not influenced by the concentration of the analyte (e.g. blood sugar). For example, if the mediator is potassium ferricyanide, the negative polarity signal applied to the working electrode WE during the second period T2 can make potassium ferricyanide not reacted with the analyte (e.g. blood sugar) reduce to potassium ferrocyanide, wherein potassium ferrocyanide is the intermediate of the present invention, and concentration of potassium ferrocyanide is not influenced by the analyte (e.g. blood sugar). But, in another embodiment of the present invention, because the electrical signal applied to the working electrode WE is a positive polarity signal during the second period T2, the majority of mediator not reacted with the analyte can generate oxidation reaction on the surface of the working electrode WE. That is to say, the high concentration oxidized state mediator can be accumulated on the surface of the working electrode WE. In addition, the present invention is not limited to the electrical signal applied to the working electrode WE being a voltage signal during the second period T2, that is, the electrical signal applied to the working electrode WE can also be a current signal during the second period T2.

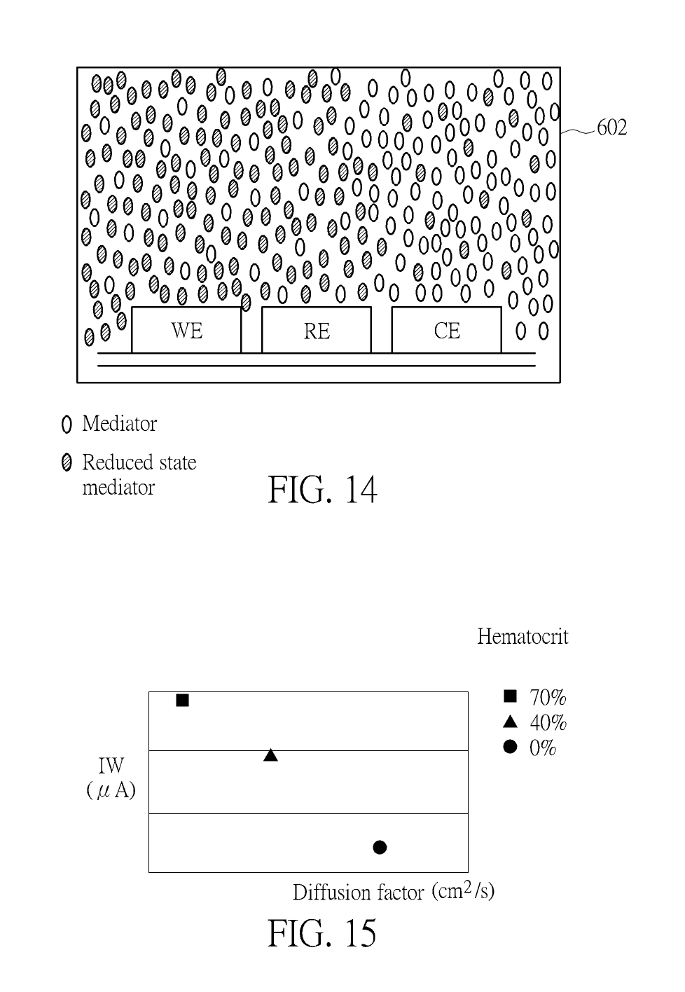

Diffusion behavior of the mediator in the sample corresponds to a diffusion factor of the sample, wherein the diffusion factor is a function corresponding to a combination of temperature, viscosity, hematocrit, lipemic and ionic strength of the sample. But, the present invention is not limited to the diffusion factor being a function corresponding to a combination of temperature, viscosity, hematocrit, lipemic and ionic strength of the sample. When the diffusion factor of the mediator in the sample is lower, the reduced state mediator (the intermediate) generated during the second period T2 cannot diffuse easily (as shown in FIG. 14); on the other hand, when the diffusion factor of the mediator in the sample is higher, the reduced state mediator (the intermediate) generated during the second period T2 can diffuse easily. Therefore, during the third period T3 in FIG. 10, when the electrical signal applied to the working electrode WE is a positive polarity signal, the working electrode WE can generate a greater current IW (second current) when the diffusion factor of the mediator in the sample is lower (because the more reduced state mediator can be accumulated on the surface of the working electrode WE, the working electrode WE can receive more electrons, resulting in the working electrode WE generating the greater current IW (second current)), and the working electrode WE can generate a smaller current IW (second current) when the diffusion factor of the mediator in the sample is higher (because the less reduced state mediator can be accumulated on the surface of the working electrode WE, the working electrode WE can receive less electrons, resulting in the working electrode WE generating the smaller current IW (second current)). In addition, in another embodiment of the present invention, the electrical signal applied to the working electrode WE during the third period T3 is a negative polarity signal opposite to a positive polarity signal during the second period T2.

In addition, during the first period T1, the second period T2, and the third period T3, the counter electrode CE is used for receiving a floating voltage VCE provided by the operational amplifier OP1 to satisfy the current IW generated by the working electrode WE. Therefore, a reaction material can be coated in a surface of the counter electrode CE (or the counter electrode CE can directly react with the electrical signal applied to the working electrode WE) to prevent a voltage of the counter electrode CE from being increased too high during the first period T1, the second period T2, and the third period T3, wherein an oxidized-reduced state of the reaction material coated in the surface of the counter electrode CE is opposite to an original oxidized-reduced state of the mediator in the sample.

In addition, as shown in FIG. 4 to FIG. 7, the reference electrode RE is coupled to a negative input terminal of the operational amplifier OP1 (that is, no current flowing through the reference electrode RE) and the reference electrode RE is between the working electrode WE and the counter electrode CE, so the reference electrode RE can prevent the reaction material coated on the surface of the counter electrode CE or a product generated by the counter electrode CE from being diffused to the working electrode WE. That is to say, the reference electrode RE can prevent the counter electrode CE from influencing the current IW generated by the working electrode WE during the first period T1, the second period T2, and the third period T3.

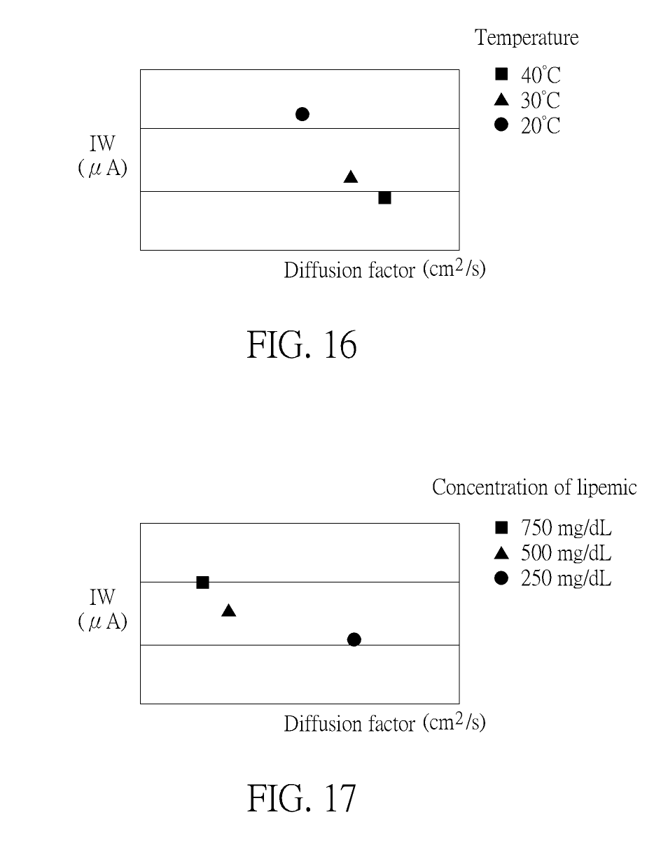

Please refer to FIG. 15 to FIG. 17. FIG. 15 to FIG. 17 are diagrams illustrating relationships between the diffusion factor of the mediator and the current IW (second current) generated by the working electrode WE under different interfering substances. As shown in FIG. 15, when hematocrit HCT of the sample is higher (the mediator has the lower diffusion factor), the current IW generated by the working electrode WE is greater. For example, the current IW generated by the working electrode WE when the hematocrit HCT of the sample is 70% is greater than the current IW generated by the working electrode WE when the hematocrit HCT of the sample is 40%. As shown in FIG. 16, when temperature of the sample is lower (the mediator has the lower diffusion factor), the current IW generated by the working electrode WE is greater. For example, the current IW generated by the working electrode WE when the temperature of the sample is 20° C. is greater than the current IW generated by the working electrode WE when the temperature of the sample is 30° C. As shown in FIG. 17, when concentration of lipemic (triglyceride) of the sample is higher (the mediator has the lower diffusion factor), the current IW generated by the working electrode WE is greater. For example, the current IW generated by the working electrode WE when the concentration of the lipemic of the sample is 750 mg/dL is greater than the current IW generated by the working electrode WE when the concentration of the lipemic of the sample is 500 mg/dL. Thus, during the third period T3 in FIG. 10, the meter 604 can calculate the diffusion factor of the intermediate (the reduced state mediator) in the sample through the current IW generated by the working electrode WE (second current) according to the above mentioned principles, wherein the second current is a diffusion current generated by the intermediate during the third period T3.

Because the concentration of the reduced state mediator on the surface of the working electrode WE is not influenced by the concentration of the analyte (e.g. blood sugar), after the diffusion factor of the mediator in the sample is generated, the meter 604 can correct an error of the concentration of the analyte in the sample to generate new concentration of the analyte according to the diffusion factor of the mediator in the sample, wherein factors causing the error of the concentration of the analyte correspond to a combination of temperature, viscosity, hematocrit, lipemic and ionic strength of the sample.

In addition, when the electrical signal applied to the working electrode WE is a voltage, a range of the electrical signal during the first period T1 and a range of the electrical signal during the third period T3 are about between 50 mV-1000 mV, the best about between 200 mV-500 mV. A range of the electrical signal during the second period T2 is about between −50 mV and −1000 mV, the best about between −100 mV and −500 mV. Further, a range of the second period T2 is about between 0.5 s and 10 s, the best about between 1 S and 8 S. In addition, in another embodiment of the present invention, the electrical signal during the second period T2 is a predetermined current.

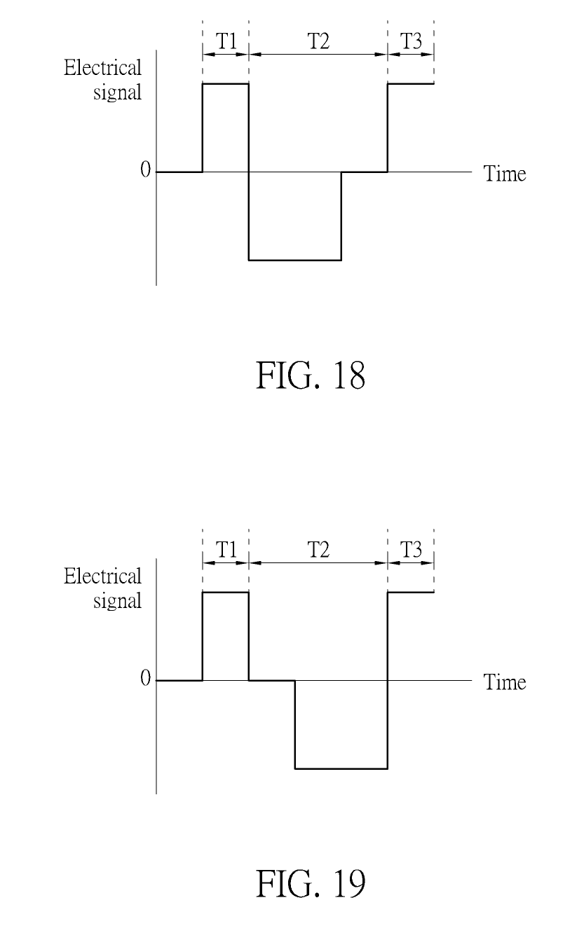

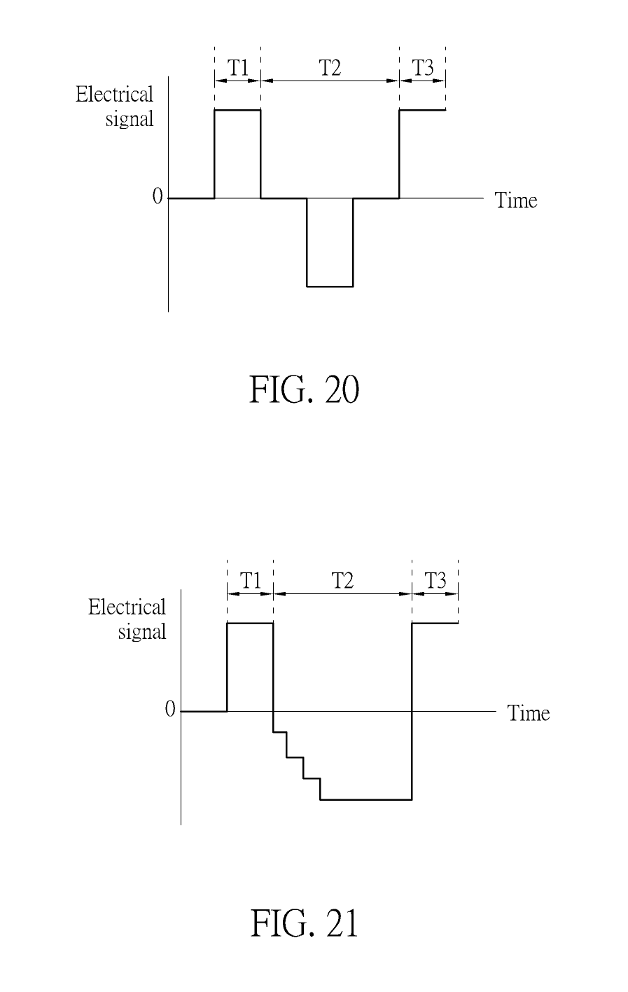

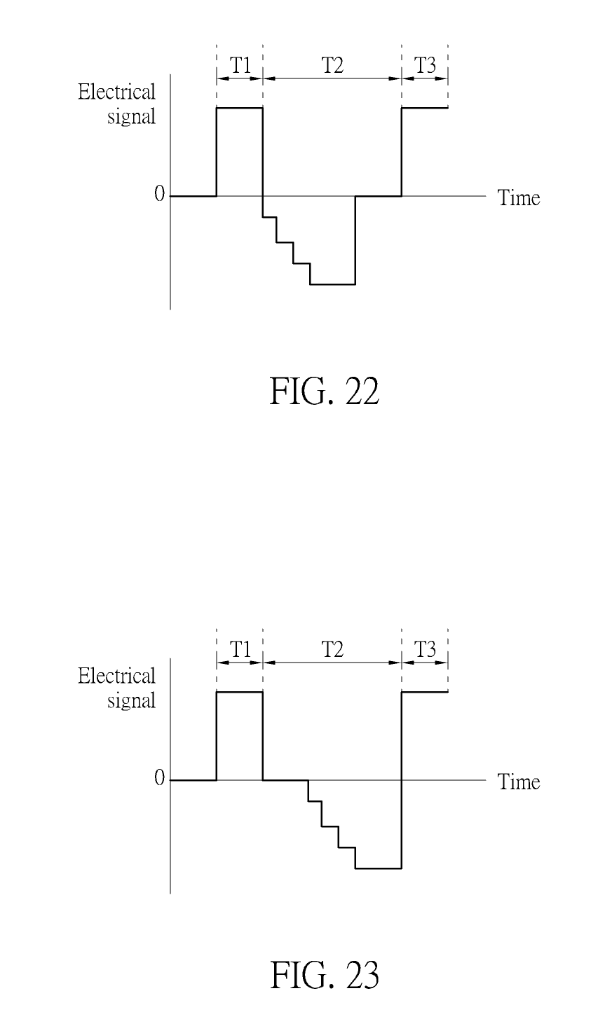

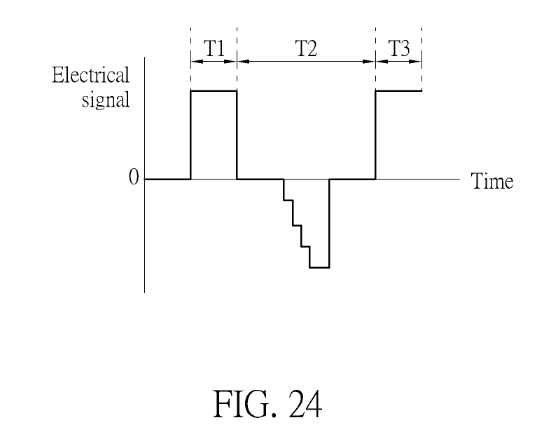

Please refer to FIG. 18 to FIG. 24. FIG. 18 to FIG. 24 are diagrams illustrating a voltage drop VWR between the working electrode WE and the reference electrode RE (equal to an electrical signal applied to the working electrode WE) during the first period T1, the second period T2, and the third period T3 according to different embodiments, wherein subsequent operational principles of the voltage drop VWR between the working electrode WE and the reference electrode RE during the first period T1, the second period T2, and the third period T3 in FIG. 18 to FIG. 24 are the same as those of the voltage drop VWR between the working electrode WE and the reference electrode RE during the first period T1, the second period T2, and the third period T3 in FIG. 10. In addition, as shown in FIG. 18 to FIG. 24, the voltage drop VWR between the working electrode WE and the reference electrode RE can be 0 (non-polarity) and a negative polarity signal during the second period T2, that is, the electrical signal applied to the working electrode WE is 0 and the negative polarity signal.

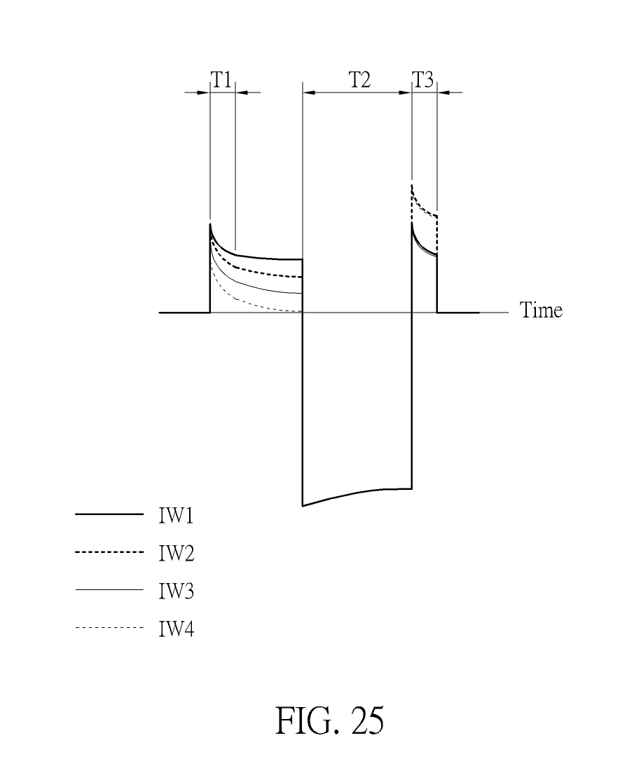

Please refer to FIG. 25. FIG. 25 is a diagram illustrating currents IW1, IW2, IW3, IW4 generated by the working electrode WE corresponding to different samples under the voltage drop VWR between the working electrode WE and the reference electrode RE in FIG. 10. Because principles of other factors (e.g. temperature, viscosity, hematocrit, lipemic and ionic strength of the sample) causing the error of the concentration of the analyte are the same as those of hematocrit, so further description thereof is omitted for simplicity. As shown in FIG. 11 and FIG. 12, when the sample (including blood sugar) is placed in the reaction region 602, the mediator in the sample can directly or indirectly seize electrons from the analyte to become the reduced state mediator, so the working electrode WE can generate the current IW (first current) through the reduced state mediator, wherein the current IW (first current) during the first period T1 can be used for calculating the initial concentration of the analyte. In addition, before the high concentration reduced state mediator is not yet generated and accumulated on the surface of the working electrode WE, the current IW (first current) generated by the working electrode WE is greater when the hematocrit of the sample is lower. Therefore, as shown in FIG. 25, during the first period T1, the current IW1 (first current) corresponding to a sample 1>the current IW2 (first current) corresponding to a sample 2>the current IW3 (first current) corresponding to a sample 3>the current IW4 (first current) corresponding to a sample 4, wherein the sample 1 has blood sugar concentration (200mg/dL) and hematocrit HCT (10%), the sample 2 has blood sugar concentration (200 mg/dL) and the hematocrit HCT (70%), the sample 3 has blood sugar concentration (100 mg/dL) and the hematocrit HCT (10%), and the sample 4 has blood sugar concentration (100mg/dL) and the hematocrit HCT (70%). As shown in FIG. 13, during the second period T2, because the electrical signal applied to the working electrode WE is the negative polarity signal, the majority of mediator not reacted with the analyte can generate reduction reaction on the surface of the working electrode WE. That is to say, the high concentration reduced state mediator (the intermediate) is accumulated on the surface of the working electrode WE. It is noted that the concentration of the reduced state mediator accumulated on the surface of the working electrode WE is not influenced by the concentration of the analyte (e.g. blood sugar), so the current IW (second current) generated by the working electrode WE is lower when the diffusion factor of the mediator in the sample is higher, and the current IW (second current) generated by the working electrode WE is higher when the diffusion factor of the mediator in the sample is lower. Therefore, during the third period T3 in FIG. 25, a difference between the current IW1 (second current) corresponding to the sample 1 and the current IW3 (second current) corresponding to the sample 3 is minor (because the sample 1 and the sample 3 have the same hematocrit HCT (10%)) and a difference between the current IW2 (second current) corresponding to the sample 2 and the current IW4 (second current) corresponding to the sample 4 is minor (because the sample 2 and the sample 4 have the same hematocrit HCT (70%)), and the current IW1 (second current) corresponding to the sample 1 and the current IW3 (second current) corresponding to the sample 3 are less than the current IW2 (second current) corresponding to the sample 2 and the current IW4 (second current) corresponding to the sample 4. Thus, during the third period T3 in FIG. 25, the meter 604 can calculate diffusion factors of the mediator in the samples 1, 2, 3, 4 respectively through the currents IW1, IW2, IW3, IW4 (second currents) generated by the working electrode according to the above mentioned principles. After the diffusion factors of the mediator in the samples 1, 2, 3, 4 are generated, the meter 604 can correct errors of concentration of analytes in the samples 1, 2, 3, 4 to generate new concentration of the analytes in the samples 1, 2, 3, 4 respectively according to the diffusion factors of the mediator in the samples 1, 2, 3, 4.

In addition, the present invention is not limited to the oxidized-reduced state of the mediator in FIG. 11 to FIG. 14. That is to say, in another embodiment of the present invention, a new mediator has an oxidized-reduced state opposite to the oxidized-reduced state of the mediator in FIG. 11 to FIG. 14, and a new electrical signal applied to the working electrode WE is opposite to the electrical signal applied to the working electrode WE in FIG. 10.

In addition, the test strips 600, 1100 and the meter 604 provided by the present invention can also be integrated into a biometric system, wherein subsequent operational principles of the biometric system can be referred to those of the test strips 600, 1100, and the meter 604, so further description thereof is omitted for simplicity.

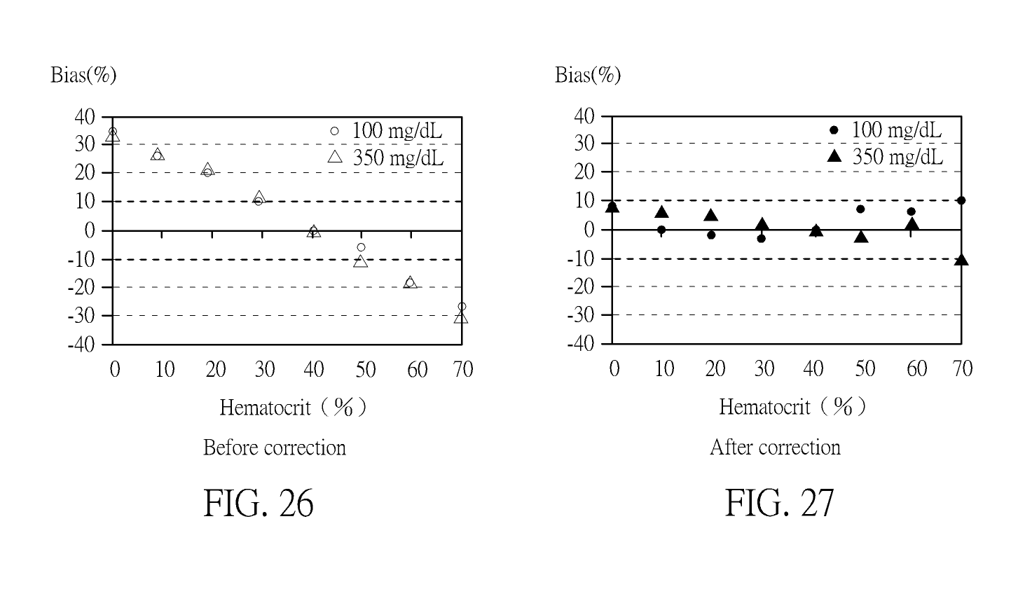

Please refer to FIG. 26. FIG. 26 is a diagram illustrating relationships between biases and concentration of analytes with different hematocrits not corrected by the present invention. As shown in FIG. 26, an analyte with 41% hematocrit is acted as a standard, wherein the biases are differences between each concentration of analyte and the standard, and the concentration of the analytes (e.g. blood sugar) in samples is 100 mg/dL and 350 mg/dL. As shown in FIG. 26, when hematocrit in the samples deviates from the standard, greater biases exist. Please refer to FIG. 27. FIG. 27 is a diagram illustrating relationships between biases and concentration of analytes with different hematocrits corrected by the present invention. As shown in FIG. 27, the above mentioned test strip and method provided by the present invention can significantly decrease biases within the whole range (0-70%) of hematocrit to control the biases away from the standard +10% to −10%.

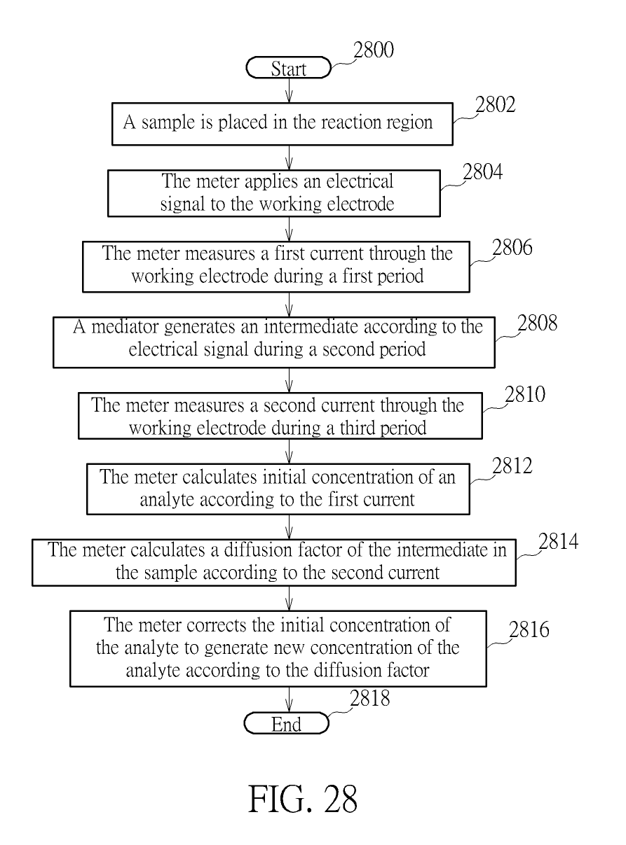

Please refer to FIG. 4, FIGS. 10-14, and FIG. 28. FIG. 28 is a flowchart illustrating a method of a test strip detecting concentration of an analyte of a sample according to another embodiment. The method in FIG. 28 is illustrated using the test strip 600 in FIG. 4. Detailed steps are as follows:

Step 2800: Start.

Step 2802: A sample is placed in the reaction region 602.

Step 2804: The meter 604 applies an electrical signal to the working electrode WE.

Step 2806: The meter 604 measures a first current through the working electrode WE during a first period T1.

Step 2808: A mediator generates an intermediate according to the electrical signal during a second period T2.

Step 2810: The meter 604 measures a second current through the working electrode WE during a third period T3.

Step 2812: The meter 604 calculates initial concentration of an analyte according to the first current.

Step 2814: The meter 604 calculates a diffusion factor of the intermediate in the sample according to the second current.

Step 2816: The meter 604 corrects the initial concentration of the analyte to generate new concentration of the analyte according to the diffusion factor.

Step 2818: End.

In Step 2802, the sample includes the analyte (e.g. blood sugar). In Step 2804, the electrical signal applied to the working electrode WE is equal to a voltage drop VWR between the working electrode WE and the reference electrode RE. As shown in FIG. 4, FIG. 10, and FIG. 11, when the sample is placed in the reaction region 602, the mediator in the sample can directly or indirectly seize electrons from the analyte to become a reduced state mediator, wherein concentration of the mediator is much greater than concentration of the analyte (e.g. the concentration of the mediator is equal to 2-4 times the concentration of the analyte). Therefore, in Step 2806, as shown in FIG. 10 and FIG. 12, during the first period T1, the electrical signal applied to the working electrode WE is a positive polarity signal, so the reduced state mediator can transfer electrons to the working electrode WE through a diffusion effect. That is to say, the working electrode WE can generate the first current through the reduced state mediator during the first period T1. But, the present invention is not limited to the mediator in the sample directly or indirectly seizing electrons from the analyte to become a reduced state mediator. That is to say, the mediator in the sample can also directly or indirectly transfer electrons to the analyte to become an oxidized state mediator.

In Step 2808, as shown in FIG. 10 and FIG. 13, because the concentration of the mediator is much greater than the concentration of the analyte, the majority of mediator not reacted with the analyte can generate reduction reaction on the surface of the working electrode WE when the electrical signal applied to the working electrode WE is a negative polarity signal during the second period T2, resulting in the high concentration reduced state mediator (that is, the intermediate) being accumulated on the surface of the working electrode WE, wherein the concentration of the reduced state mediator accumulated on the surface of the working electrode WE is not influenced by the concentration of the analyte (e.g. blood sugar). But, in another embodiment of the present invention, because the electrical signal applied to the working electrode WE is a positive polarity signal during the second period T2, the majority of mediator not reacted with the analyte can generate oxidation reaction on the surface of the working electrode WE. That is to say, the high concentration oxidized state mediator can be accumulated on the surface of the working electrode WE. In addition, the present invention is not limited to the electrical signal applied to the working electrode WE being a voltage signal during the second period T2, that is, the electrical signal applied to the working electrode WE can also be a current signal during the second period T2.

In Step 2810, diffusion behavior of the mediator in the sample corresponds to the diffusion factor of the sample, wherein the diffusion factor is a function corresponding to a combination of temperature, viscosity, hematocrit, lipemic and ionic strength of the sample. But, the present invention is not limited to the diffusion factor being a function corresponding to a combination of temperature, viscosity, hematocrit, lipemic and ionic strength of the sample. When the diffusion factor of the mediator in the sample is lower, the reduced state mediator generated during the second period T2 cannot diffuse easily (as shown in FIG. 14); on the other hand, when the diffusion factor of the mediator in the sample is higher, the reduced state mediator generated during the second period T2 can diffuse easily. Therefore, during the third period T3 in FIG. 10, when the electrical signal applied to the working electrode WE is a positive polarity signal, the working electrode WE can generate the greater second current when the diffusion factor of the mediator in the sample is lower (because the more reduced state mediator can be accumulated on the surface of the working electrode WE, the more electrons can be received by the working electrode WE, resulting in the working electrode WE generating the greater second current), and the working electrode WE can generate the smaller second current when the diffusion factor of the mediator in the sample is higher (because the less reduced state mediator can be accumulated on the surface of the working electrode WE, the fewer electrons can be received by the working electrode WE, resulting in the working electrode WE generating the smaller second current). In addition, in another embodiment of the present invention, the electrical signal applied to the working electrode WE during the third period T3 is a negative polarity signal opposite to a positive polarity signal during the second period T2.

In Step 2812, the first current during the first period T1 can be used for calculating the initial concentration of the analyte. In addition, in Step 2814, because diffusion behavior of the mediator in the sample corresponds to the diffusion factor of the sample, the meter 604 can calculate the diffusion factor of the intermediate (the reduced state mediator) in the sample according to the second current. Finally, in Step 2816, after the diffusion factor is generated, the meter 604 can correct the initial concentration of the analyte to generate the new concentration of the analyte according to the diffusion factor.

In addition, during the first period T1, the second period T2, and the third period T3, the counter electrode CE is used for receiving a floating voltage VCE provided by the operational amplifier OP1 to satisfy the first current and the second current generated by the working electrode WE. Therefore, a reaction material can be coated on the surface of the counter electrode CE (or the counter electrode CE can directly react with the electrical signal applied to the working electrode WE) to prevent a voltage of the counter electrode CE from being increased too high during the first period T1, the second period T2, and the third period T3, wherein an oxidized-reduced state of the reaction material coated on the surface of the counter electrode CE is opposite to an original oxidized-reduced state of the mediator in the sample.

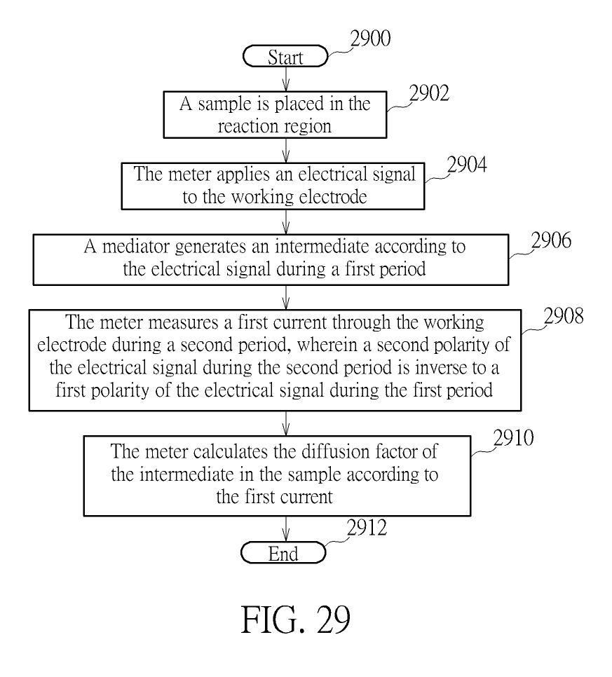

Please refer to FIG. 4, FIG. 10, and FIG. 29. FIG. 29 is a flowchart illustrating a method of utilizing a test strip to detect diffusion factor of a mediator in a sample according to another embodiment. The method in FIG. 29 is illustrated using the test strip 600 in FIG. 4. Detailed steps are as follows:

Step 2900: Start.

Step 2902: A sample is placed in the reaction region 602.

Step 2904: The meter 604 applies an electrical signal to the working electrode WE.

Step 2906: A mediator generates an intermediate according to the electrical signal during a first period.

Step 2908: The meter 604 measures a first current through the working electrode WE during a second period, wherein a second polarity of the electrical signal during the second period is inverse to a first polarity of the electrical signal during the first period;

Step 2910: The meter 604 calculates the diffusion factor of the intermediate in the sample according to the first current.

Step 2912: End.

A difference between the embodiment in FIG. 29 and the embodiment in FIG. 28 is that in Step 2906, the mediator generates the intermediate during the first period (corresponding to the second period T2 in FIG. 10) according to the electrical signal; in Step 2908, the meter 604 measures the first current (corresponding to the second current in the embodiment in FIG. 28) through the working electrode WE during the second period (corresponding to the third period T3 in FIG. 10) ; and in Step 2910, the meter 604 calculates the diffusion factor of the intermediate in the sample according to the first current (corresponding to the second current in the embodiment in FIG. 28). Therefore, any configuration which utilizes a second polarity of an electrical signal during a second period inverse to a first polarity of the electrical signal during a first period to detect a diffusion factor of a mediator in a sample falls within the scope of the present invention.

To sum up, the method of a test strip detecting concentration of an analyte of a sample and the test strip with three electrodes utilize the working electrode to generate a first current for calculating initial concentration of the analyte of the sample according to an electrical signal provided by the meter during a first period, utilize the working electrode to make a mediator in the sample generate reaction according to the electrical signal provided by the meter during a second period, and utilize the working electrode to generate a second current for calculating a diffusion factor of the mediator in the sample according to the electrical signal provided by the meter during a third period. After the diffusion factor of the mediator in the sample is generated, the meter can correct the initial concentration of the analyte in the sample to generate new concentration of the analyte according to the diffusion factor of the mediator in the sample. Therefore, compared to the prior art, the present invention can accurately correct the initial concentration of the analyte in the sample. In addition, the method of utilizing a test strip to detect a diffusion factor of a mediator in a sample further provided by the present invention utilizes a second polarity of an electrical signal during a second period inverse to a first polarity of the electrical signal during a first period to detect the diffusion factor of the mediator. Therefore, compared to the prior art, the present invention can rapidly, simply, and accurately detect the diffusion factor of the mediator.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method of a test strip detecting concentration of an analyte of a sample, wherein the test strip comprises a substrate and a reaction region, the reaction region comprises a working electrode, a reference electrode, and a counter electrode, and an enzyme is coated in the reaction region, the method comprising:
placing the sample in the reaction region, wherein the analyte reacts with the enzyme to generate a plurality of electrons, and the plurality of electrons are transferred to the working electrode through a mediator;
applying an electrical signal to the working electrode;
measuring a first current through the working electrode during a first period;
the mediator generating an intermediate according to the electrical signal during a second period;
measuring a second current through the working electrode during a third period, wherein a second polarity of the electrical signal during the second period is inverse to a first polarity of the electrical signal during the first period and a third polarity of the electrical signal during the third period;
calculating initial concentration of the analyte according to the first current;
calculating a diffusion factor of the intermediate in the sample according to the second current; and
correcting the initial concentration to generate new concentration of the analyte according to the diffusion factor.

2. The method of claim 1, wherein when the electrical signal is a voltage, a range of the electrical signal during the first period and a range of the electrical signal during the third period are between 50 mV-1000 mV, a range of the electrical signal during the second period is between −50 mV-1000 mV, and a range of the second period is between 0.5 s-10 s.

3. The method of claim 1, wherein the sample at least covers the working electrode.

4. The method of claim 1, wherein when the sample is placed in the reaction region, the reference electrode receives a reference voltage, and when the electrical signal is a voltage, the electrical signal is equal to a voltage difference between a voltage of the working electrode and the reference voltage.

5. The method of claim 1, wherein the sample is placed in the reaction region, the counter electrode receives a floating voltage to satisfy a current generated by the working electrode during the first period, the second period, and the third period.

6. The method of claim 1, wherein the intermediate is a reduced state mediator or an oxidized state mediator.

7. The method of claim 1, wherein the second current is a diffusion current generated by the intermediate during the third period.

8. The method of claim 1, wherein the second period is behind the first period, and the third period is behind the second period.

9. The method of claim 1, wherein the electrical signal is a predetermined current during the second period.

10. The method of claim 1, wherein the diffusion factor corresponds to a combination of temperature, viscosity, hematocrit, lipemic and ionic strength of the sample.

11. The method of claim 1, wherein the mediator is coated in the reaction region.

12. The method of claim 1, wherein the mediator is added to the reaction region when the sample is placed in the reaction region.

13. A test strip with three electrodes, the test strip comprising:
a substrate; and
a reaction region formed on a first end of the substrate, and an enzyme is coated in the reaction region, wherein when a sample is placed in the reaction region, an analyte reacts with the enzyme to generate a plurality of electrons, and the plurality of electrons are transferred through a mediator, the reaction region comprising:
a working electrode for receiving an electrical signal when the sample is placed in the reaction region, generating a first current according to the electrical signal during a first period, and generating a second current according to the electrical signal during a third period behind a second period, wherein a second polarity of the electrical signal during the second period is inverse to a first polarity of the electrical signal during the first period and a third polarity of the electrical signal during the third period, wherein the mediator generates an intermediate according to the electrical signal during the second period;
a reference electrode for receiving a reference voltage when the sample is placed in the reaction region; and
a counter electrode for receiving a floating voltage when the sample is placed in the reaction region to satisfy a current generated by the working electrode during the first period, the second period, and the third period, wherein the current comprises the first current and the second current;
wherein the first current is used for calculating initial concentration of the analyte, the second current is used for calculating a diffusion factor of the intermediate, and the diffusion factor is used for correcting the initial concentration to generate new concentration of the analyte.

14. The test strip of claim 13, wherein the electrical signal is a predetermined current during the second period.

15. The test strip of claim 13, wherein the working electrode is connected to a first pad and a second pad, wherein the second pad is used for transmitting the electrical signal to the working electrode, and for stabilizing the working electrode at a corresponding voltage according to the electrical signal, wherein the first pad is used for transmitting the current generated by the working electrode during the first period, the second period, and the third period, wherein the first pad and the second pad are formed on a second end of the substrate, and the second end of the substrate is opposite to the first end of the substrate.

16. The test strip of claim 15, wherein the reference electrode is connected to a third pad, and the counter electrode is connected to a fourth pad, wherein the third pad is used for transmitting the reference voltage to the reference electrode, and the fourth pad is used for transmitting the floating voltage to the counter electrode, wherein the third pad and the fourth pad are formed on the second end of the substrate.

17. A method of a test strip detecting concentration of an analyte of a sample, wherein the test strip comprises a substrate and a reaction region, the reaction region comprises a working electrode, a reference electrode, and a counter electrode, and an enzyme is coated in the reaction region, the method comprising:
placing the sample in the reaction region, wherein the analyte reacts with the enzyme to generate a plurality of electrons, and the plurality of electrons are transferred to the working electrode through a mediator;
applying an electrical signal to the working electrode;
measuring a first current through the working electrode during a first period;
the mediator generating an intermediate according to the electrical signal during a second period;
measuring a second current through the working electrode during a third period, wherein the electrical signal has a second polarity and a non-polarity during the second period, and the second polarity is inverse to a first polarity of the electrical signal during the first period and a third polarity of the electrical signal during the third period;
calculating initial concentration of the analyte according to the first current;
calculating a diffusion factor of the intermediate in the sample according to the second current; and
correcting the initial concentration to generate new concentration of the analyte according to the diffusion factor.

18. A method of utilizing a test strip to detect a diffusion factor of a mediator in a sample, wherein the test strip comprises a reaction region, and the reaction region comprises a working electrode, a reference electrode, and a counter electrode, the method comprising:
placing the sample in the reaction region;
applying an electrical signal to the working electrode;
the mediator generating an intermediate according to the electrical signal during a first period;
measuring a first current through the working electrode during a second period behind the first period, wherein a second polarity of the electrical signal during the second period is inverse to a first polarity of the electrical signal during the first period; and
calculating the diffusion factor of the intermediate in the sample according to the first current.

19. The method of claim 18, wherein the electrical signal is a voltage, a range of the electrical signal during the second period is between 50 mV-1000 mV, a range of the electrical signal during the first period is between −50 mV-1000 mV, and a range of the first period is between 0.5 s-10 s.

20. The method of claim 18, wherein when the sample is placed in the reaction region, the reference electrode receives a reference voltage, and when the sample is placed in the reaction region, the counter electrode receives a floating voltage to satisfy a first current generated by the working electrode during the first period and the second period.

21. The method of claim 18, wherein the first current is a diffusion current generated by the intermediate during the second period.

22. The method of claim 18, wherein the electrical signal is a predetermined current during the first period.

23. A method of utilizing a test strip to detect a diffusion factor of a mediator in a sample, wherein the test strip comprises a reaction region, and the reaction region comprises a working electrode, a reference electrode, and a counter electrode, the method comprising:
placing the sample in the reaction region;
applying an electrical signal to the working electrode;
the mediator generating an intermediate according to the electrical signal during a first period;
measuring a first current through the working electrode during a second period behind the first period, wherein the electrical signal has a first polarity and a non-polarity during the first period, and the first polarity is inverse to a second polarity of the electrical signal during the second period; and
calculating the diffusion factor of the intermediate in the sample according to the first current.
